(19) United States
(12) Patent Application Publication
(10) Pub. No.: US 2014/0073970 A1
(43) Pub. Date: Mar. 13, 2014

(54) Physiological Condition Monitor

(71) Applicant: ICON Health & Fitness, Inc., Logan, UT (US)

(72) Inventor: Darren C. Ashby, Richmond, UT (US)

(21) Appl. No.: 14/025,821

(22) Filed: Sep. 12, 2013

Related U.S. Application Data
(60) Provisional application No. 61/700,379, filed on Sep. 13, 2012.

(51) Int. Cl. A61B 5/083

(52) U.S. Cl. CPC A61B 5/0833
USPC 600/484

(57) ABSTRACT

A physiological condition monitor that is usable in determining a user's oxygen consumption (VO₂) includes a heart rate monitor that monitors a user's heart rate, a respiration monitor, and a processor. The respiration monitor monitors how much a circumference of the user's chest changes as the user breaths. The respiration monitor may include a material that has electrical characteristics that change as the material expands and contracts. The changes in the electrical characteristics may be proportional to the changes in the circumference of the user's chest. The processor uses the changes in the circumference of the user's chest to approximate the volume of air the user inhales as the user breaths. Using the volume approximation and the user's heart rate, the processor determines the user's level of oxygen consumption (VO₂).

### CROSS-REFERENCE TO RELATED APPLICATIONS

This application claims priority to U.S. Provisional Patent Application No. 61/700,379 filed on Sep. 13, 2012.

### TECHNICAL FIELD

This disclosure relates generally to systems, methods, and devices for monitoring and determining physiological conditions of an individual. More particularly, the disclosure relates to a monitoring device wearable by an individual which can monitor various physiological conditions of the individual and determine the individual's oxygen consumption (VO₂) level.

### BACKGROUND

Over the last four decades, the prevalence of obesity and weight-related ailments has increased dramatically. Fortunately, public awareness of the causes and effects of being overweight has increased, and many people are not only learning about how the body uses fat, but are also making dramatic lifestyle changes. As part of that public awareness, people are becoming more educated about the importance of proper nutrition and exercise, including cardiovascular training.

Certain physiological conditions, including heart rate and respiration, are indicative of an individual's cardiovascular and overall fitness levels. Various monitoring systems and devices have been developed to detect heart rate and/or respiration. For instance, U.S. Pat. No. 4,960,118 discloses a chest strap apparatus for measuring respiratory flow for a user. In particular, the chest strap includes a series of piezoelectric film strips that are stressed as the user breaths. The stresses on the films produce electric outputs that may be used to determine the user's respiratory flow rate. Similarly, U.S. Pat. No. 7,740,588, U.S. Pat. No. 7,643,873, U.S. Pat. No. 4,889,131, and U.S. Pat. No. 4,576,179 disclose monitoring devices that may be worn around a user's chest and which detect respiration data, such as respiration rate, for the user. Additionally, these monitoring devices also include heart rate monitors for detecting the user's heart rate.

### SUMMARY OF THE INVENTION

In one aspect of the disclosure, a physiological condition monitor includes a heart rate monitor, a respiration monitor, and a processor. The heart rate monitor monitors a user's heart rate. The respiration monitor monitors how much a circumference of the user's chest changes as the user breaths. The processor uses the changes in the circumference of the user's chest to approximate the volume of air the user inhales and exhales as the user breaths. Further, the processor uses the volume approximation and the user's heart rate to determine the user's level of oxygen consumption (VO₂).

Another aspect of the disclosure that may be included in any combination with other aspects disclosed herein may include the physiological condition monitor including one or more straps that selectively secure the physiological condition monitor around the chest of the user.

Another aspect of the disclosure that may be included in any combination with other aspects disclosed herein may include the respiration monitor comprising an elastic material that extends around at least a portion of the circumference of the user's chest.

Another aspect of the disclosure that may be included in any combination with other aspects disclosed herein may include the elastic material comprising rubber.

Another aspect of the disclosure that may be included in any combination with other aspects disclosed herein may include the elastic material being impregnated or doped with at least one of carbon or silicone.

Another aspect of the disclosure that may be included in any combination with other aspects disclosed herein may include the respiration monitor being able to expand and contract as the user inhales and exhales.

Another aspect of the disclosure that may be included in any combination with other aspects disclosed herein may include an electrical resistance of the respiration monitor being proportional to the length of the respiration monitor.

Another aspect of the disclosure that may be included in any combination with other aspects disclosed herein may include the electrical resistance of the respiration monitor changing as the respiration monitor expands and contracts.

Another aspect of the disclosure that may be included in any combination with other aspects disclosed herein may include the electrical resistance changing in proportion to how much the circumference of the user's chest changes as the user breaths.

Another aspect of the disclosure that may be included in any combination with other aspects disclosed herein may include the physiological condition monitor including a temperature sensor that detects the user's temperature.

Another aspect of the disclosure that may be included in any combination with other aspects disclosed herein may include the processor using the user's temperature to determine the user's level of oxygen consumption (VO₂).

Another aspect of the disclosure that may be included in any combination with other aspects disclosed herein may include the heart rate monitor comprising an electrocardiogram (ECG) sensor.

Another aspect of the disclosure that may be included in any combination with other aspects disclosed herein may include the heart rate monitor comprising a light emitting sensor.

Another aspect of the disclosure that may be included in any combination with other aspects disclosed herein may include the physiological condition monitor including a wireless transmitter.

Another aspect of the disclosure that may be included in any combination with other aspects disclosed herein may include the physiological condition monitor including a body motion monitor.

Another aspect of the disclosure that may be included in any combination with other aspects disclosed herein may include the body motion monitor comprising at least one of a pedometer, an accelerometer, and a gyroscope.

Another aspect of the disclosure that may be included in any combination with other aspects disclosed herein may include the respiration monitor including a stretchable material.

Another aspect of the disclosure that may be included in any combination with other aspects disclosed herein may include the stretchable material having an electrical characteristic that changes as the stretchable material expands and contracts.

Another aspect of the disclosure that may be included in any combination with other aspects disclosed herein may include that the changes in the electrical characteristic of the stretchable material are proportional to the changes in the circumference of the user's chest.

Another aspect of the disclosure that may be included in any combination with other aspects disclosed herein may include the stretchable material comprising rubber impregnated or doped with at least one of carbon or silicone.

Another aspect of the disclosure that may be included in any combination with other aspects disclosed herein may include a method for determining a person's oxygen consumption (VO₂) that includes determining a heart rate of the person.

Another aspect of the disclosure that may be included in any combination with other aspects disclosed herein may include a method that method includes measuring a change in a chest circumference of the person.

Another aspect of the disclosure that may be included in any combination with other aspects disclosed herein may include a method that includes approximating a volume of air breathed by the person.

Another aspect of the disclosure that may be included in any combination with other aspects disclosed herein may include approximating a volume of air breathed by a person including using the measurement of the change in the chest circumference of the person.

Another aspect of the disclosure that may be included in any combination with other aspects disclosed herein may include measuring the change in the chest circumference of the person comprises measuring a change in length of a respiration monitor.

Another aspect of the disclosure that may be included in any combination with other aspects disclosed herein may include measuring the change in length of a respiration monitor comprises detecting a change in an electrical characteristic of the respiration monitor.

Another aspect of the disclosure that may be included in any combination with other aspects disclosed herein may include a method that includes processing the heart rate and the volume approximation to determine the person's oxygen consumption (VO₂).

### BRIEF DESCRIPTION OF THE DRAWINGS

FIG. 1 illustrates a physiological condition monitor being worn by a user according to one example embodiment of the present disclosure.

FIG. 2 illustrates a perspective view of the physiological condition monitor of FIG. 1.

FIG. 3 is schematic diagram of the physiological condition monitor of FIGS. 1 and 2.

FIG. 4 is a functional block diagram of a process for monitoring a physiological condition.

FIG. 5 is a functional block diagram of a process for determining an exercise efficiency.

### DETAILED DESCRIPTION

The present disclosure is directed to systems, methods, and devices for monitoring and determining physiological conditions of a user. More specifically, the present disclosure relates to systems, methods, and devices that detect certain physiological conditions of a user and determine the user's oxygen consumption (VO₂) level.

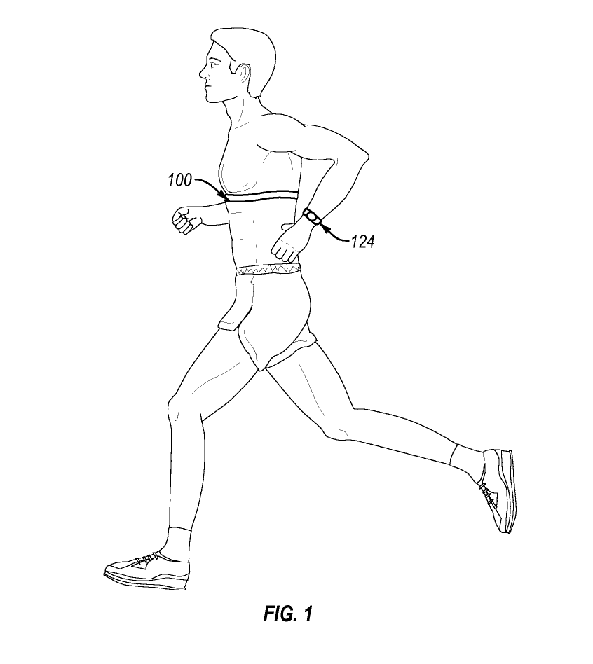

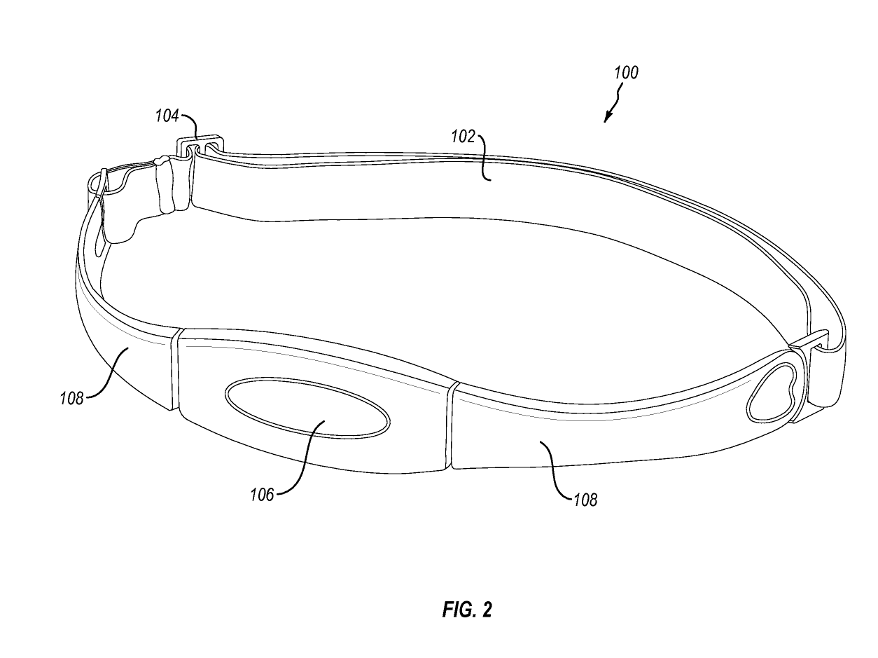

Depicted in FIGS. 1 and 2 is a representation of one illustrative physiological condition monitor 100 (also referred to herein as monitor 100), which may incorporate the novel features of the present invention, including various novel devices, functionalities, hardware and software modules, and the like. As shown, monitor 100 is designed to be worn around a user's trunk region, and typically around the user's chest.

Monitor 100 includes a strap 102 and a fastener 104 for securing monitor 100 around the user. Strap 102 may be formed of various materials. For instance, strap 102 may be formed of materials that are stretchable or have elastic properties. Alternatively, strap 102 may be formed of materials that are generally inelastic. Fastener 104 may be used to secure strap 102 around the user or adjust the size of strap 102, and may take various forms. For instance, fastener 104 may include a clip that selectively secures two ends of strap 102 together. Likewise, fastener 104 may include hook and loop fabrics that selectively secure two ends of strap 102 together. Still further, fastener 104 may be a slidable ring that selectively adjusts the length of strap 104, and thus the circumference of monitor 100.

Monitor 100 also includes a heart rate monitor 106 that can detect the user's heart rate. Heart rate monitor 106 may take any of a number of forms. By way of non-limiting example, heart rate monitor 106 may take the form of a light emitting sensor. The light emitting sensor may include a light emitter, such as an infrared LED, that is able to illuminate the user's skin to a predetermined depth. Additionally, the light emitting sensor may also include one or more photo detectors that detect the light reflected by the user. Based on differences in the emitted and detected light, the user's heart rate can be determined.

Heart rate monitor 106 may also take the form of an electrocardiogram (ECG) sensor. The ECG sensor may include a pair of electrodes that can be positioned against or adjacent to the user's skin. The electrodes may detect the electrical activity of the user's heart, from which the user's heart rate can be determined.

Monitor 100 may also include one or more respiration monitors 108. The embodiment illustrated in FIG. 2 includes two respiration monitors 108 that extend from opposing ends of heart rate monitor 106 and connect to strap 102. In the illustrated embodiment, the two respiration monitors 108 comprise approximately one third of the circumference of monitor 100.

It is understood, however, that the embodiment illustrated in FIG. 2 is merely one example. For instance, the two respiration monitors 108 may comprise more or less than one third of the circumference of monitor 100. Additionally, monitor 100 may include one or more respiration monitors 108. For instance, monitor 100 may include a single respiration monitor 108. The single respiration monitor 108 may extend around all or a portion of the circumference of monitor 100.

Regardless of the number of respiration monitors used or their individual lengths, each respiration monitor 108 may be designed to monitor expansion and/or contraction thereof. By way of example, each respiration monitor 108 may comprises an elastic material that can expand and contract. The elastic material may have certain electrical properties that allow for the extent of the expansion and/or contraction to be determined.

For instance, the electrical resistance of the elastic material may be related to the length of thereof. In other words, the electrical resistance of the elastic material may change as the elastic material expands and contracts. Furthermore, the changes in resistance may be related to the degree of expansion and/or contraction of the elastic material. By way of example, the resistance of the elastic material may be proportional to the length of thereof. Accordingly, when the elastic material expands or contracts a predetermined amount, the resistance thereof will increase or decrease a proportional amount. One example of an elastic material suitable for respiration monitor 108 is a rubber that is doped or impregnated with materials such as silicone and carbon.

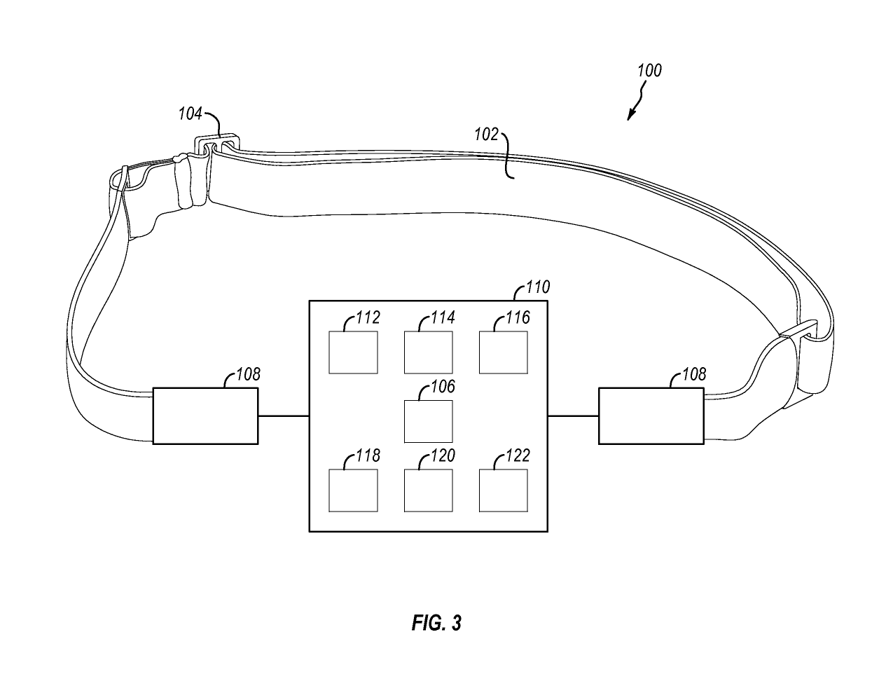

With continued attention to FIGS. 1 and 2, attention is now directed to FIG. 3, which illustrates a partial block diagram of monitor 100. As shown in FIG. 3, heart rate monitor 106 is incorporated into a control unit 110. In addition to heart rate monitor 106, control unit 110 also includes a battery 112, a controller 114, a memory 116, a transmitter 118, a temperature sensor 120, and a body motion monitor 122. Each of the components of control unit 110 may be in communication with one or more of the other components of control unit 110 and/or respiration monitors 108. Although heart rate monitor 106, battery 112, controller 114, memory 116, and transmitter 118 are illustrated as a collection of individual components that form control unit 110, one or more of these components may be separated from one or more of the other components of control unit 110. Similarly, one or more of the illustrated components of control unit 110 may be combined together.

Battery 112 may provide power to the various components of monitor 100, including the other components of control unit 110. Additionally, battery 112 may provide electrical current to respiration monitors 108. The electrical current that passes through respiration monitors 108 can be used to determine the electrical resistance of respiration monitors 108. As noted above, the electrical resistance of respiration monitors 108 can be used to determine how far respiration monitors 108 have expanded or contracted.

Controller 114 may take the form of a computer, a processor, a microprocessor, a microcontroller, state machine or other similar device. Controller 114 may control the operation of one or more features of monitor 100. Additionally, controller 114 may analyze and/or process the data detected by heart rate monitor 106, respiration monitors 108, temperature sensor 120, and/or body motion monitor 122. Furthermore, controller 114 may cause memory 116 to store and/or may cause transmitter 118 to communicate to a separate device the collected and/or processed data.

Transmitter 118 may communicate the collected and/or processed data to a separate device via a wireless connection. For instance, transmitter 118 may communicate, via the wireless connection, the collected and/or processed data to watch 124 shown in FIG. 1. Similarly, transmitter 118 may communicate, via the wireless connection, the collected and/or processed data to another electronic device, such as a smartphone or computer. The wireless connection may be any type of wireless connection, including Bluetooth, infrared (IR), radio frequency (RF), wireless fidelity (Wi-Fi), and the like. Accordingly, transmitter 118 may be a Bluetooth, infrared (IR), radio frequency (RF), wireless fidelity (Wi-Fi), or other type of wireless transmitter. Additionally, although not illustrated, monitor 100 may be configured for a wired connection to another electronic device.

Body motion monitor 122 may detect the movements of the user's body. In one embodiment, body motion monitor 122 primarily detects the movement of the user's trunk region. For instance, body motion monitor 122 may detect the vertical movements of the user's trunk region and/or the user's core body impact. This data may be useful in determining the user's running efficiency, for example. Body motion monitor 122 may also detect horizontal and/or lateral movements of the user's body. Depending on the type and amount of data desired, body motion monitor 122 may take the form of a pedometer, an accelerometer, a gyroscope, or the like.

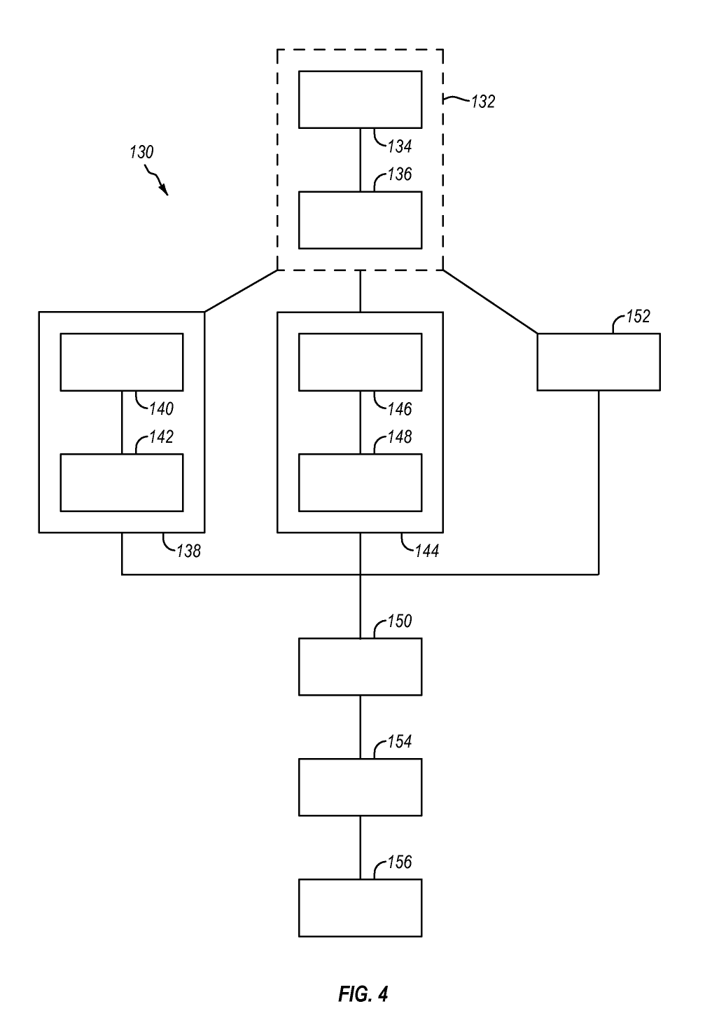

Attention is now directed to FIG. 4, which illustrates a flow diagram of an exemplary method 130 that may be implemented to monitor one or more physiological conditions of a user. Method 130 may optionally begin with step 132 in which a physiological condition monitor (e.g. physiological condition monitor 100) is associated with a user and the physiological condition monitor is calibrated. More specifically, step 132 may include securing the physiological condition monitor around the chest of a user (at step 134). Once the physiological condition monitor is secured around the user, step 132 may also include calibrating one or more respiration monitors (e.g., respiration monitors 108) of the physiological condition monitor (at step 136).

Calibration of the respiration monitors may include detecting minimum and maximum circumferences of the user's chest. The minimum chest circumference may be detected when the user has completely exhaled. Likewise, the maximum circumference may be detected when the user has completely inhaled. The calibration process may also include collecting inhale and exhale volume readings for the user using a separate inhale/exhale volume meter. The collected inhale and exhale volume readings may be input into an electronic device (e.g., smartphone, computer, watch 124) and either stored or communicated to the physiological condition monitor for later use.

Method 130 may also include (at step 138) monitoring the user's heart rate. Monitoring the user's heart rate may include collecting data (at step 140) regarding the electrical activity of the user's heart or collecting data regarding light reflected from the user as discussed herein. Additionally, monitoring the user's heart rate may also include processing (at step 142) the data collected in step 140 to determine the user's heart rate.

At generally the same time the user's heart rate is being monitored, the user's respiration is also monitored (at step 144). Monitoring the user's respiration may include detecting changes in the circumference of the user's chest (at step 146). Detecting the changes in the circumference of the user's chest may include detecting the rate at which the chest circumference changes, how much the chest circumference changes, and/or the maximum and minimum circumference sizes for some or all of the inhale/exhale cycles. This may be accomplished by passing a current through the respiration monitor and detecting changes in the resistance in the respiration monitor. Since the resistance level of the respiration monitor is related to the length of the respiration monitor, detecting the changes in the resistance level allows for the changes in the length of the respiration monitor to be determined. The length changes in the respiration monitor can then be used to determined changes in the chest circumference.

After collection, the respiration data may be processed (at step 148). Processing the data about the rate at which the chest circumference changed may provide an approximation for the user's respiration rate. Similarly, processing the data regarding the extent to which the chest circumference changed, and/or the maximum and minimum circumference sizes for the inhale/exhale cycles may provide approximations regarding how much the volume of the user's chest cavity increased and decreased during each inhale/exhale cycle. Based upon the changes in volume of the user's chest cavity, an approximation of the volume of air the user inhaled and exhaled during each inhale/exhale cycle may be determined.

In step 150 of method 130, the user's level of oxygen consumption (VO₂) is determined. More specifically, using the user's heart rate and respiration data, including one or more of the user's respiration rate, how much the chest circumference changed, and the volumes of air inhaled and exhaled, an approximation of the user's VO₂ levels can be determined.

Method 130 may also optionally include detecting the user's temperature (at step 152) at generally the same time as the user's heart rate and respiration are being monitored. The user's detected temperature data may also be used in step 150 in determining the user's VO₂ levels.

Still further, method 130 may also optionally include transmitting (at step 154) one or more of the user's heart rate data, respiration data, and VO₂ levels to another electronic device, such as a smartphone, computer, or watch (e.g. watch 124), which can display (step 156) some or all of the data to the user.

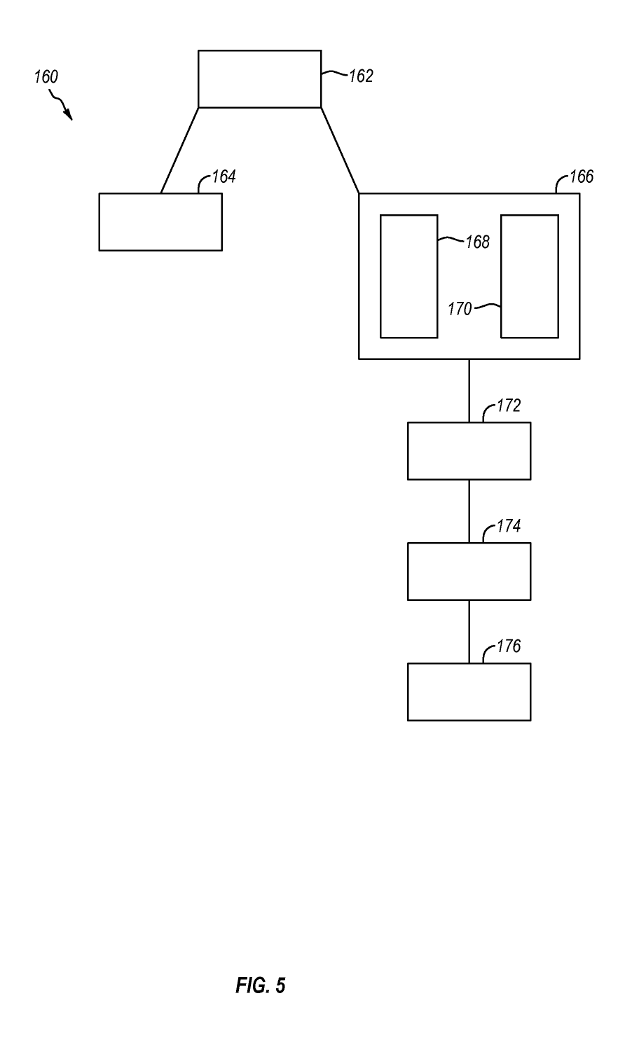

Attention is now directed to FIG. 5, which illustrates a flow diagram of an exemplary method 160 that may be implemented to monitor a user's body motions and determine an exercise efficiency for the user. Method 160 may begin with step 162 in which a body motion monitor (e.g. body motion monitor 122) is associated with a user. As discussed herein, the body motion monitor may be secured around the chest of the user as part of a physiological condition monitor.

Once the body motion monitor is secured around the user, the user may perform an exercise, such as running, in step 164. While the user is performing the exercise, the body motion monitor monitors the motions of the user's body in step 166. For instance, monitoring the motions of the user's body may include monitoring vertical movements of the user's trunk region (step 168). Monitoring the motions of the user's body may also or alternatively include monitoring the user's core body impact levels (e.g., how hard the user is hitting the ground) (step 170).

During or at the conclusion of the exercise, the data collected during step 166, including the data collected during one or both of steps 168, 170, is processed (at step 172) to determine an exercise efficiency for the user. The exercise efficiency may indicate how smoothly the user is running, whether excessive energy is being expended in vertical movements rather than horizontal movements, and the like.

Still further, method 160 may also optionally include transmitting (at step 174) the user's exercise efficiency data to another electronic device, such as a smartphone, computer, or watch (e.g. watch 124), which can display (step 176) the data to the user.

### INDUSTRIAL APPLICABILITY

In general, embodiments of the present disclosure relate to exercise systems, devices, and methods that allow for one or more physiological conditions of a user to be detected, monitored, and/or determined in a noninvasive and unobtrusive manner. The one or more physiological conditions may include the user's heart rate, temperature, body motions, and respiration. The physiological conditions that are detected or monitored may be used individually or in combination to determine other information about the user, including the volume of air inhaled and/or exhaled, oxygen consumption (VO₂), and exercise efficiency.

The systems and devices of the present disclosure may include one or more sensors or monitors for collecting data regarding the one or more desired physiological conditions. The one or more sensors or monitors may detect the one or more desired physiological conditions directly. For instance, a temperature sensor may directly detect the user's body temperature. Alternatively, the one or more sensors or monitors may detect or monitor the one or more desired physiological conditions indirectly. For instance, a heart rate monitor may detect electrical activity of the heart or properties of light reflected by the user. The electrical activity of the heart or properties of the reflected light may then be used to determine the user's heart rate.

As noted, the systems and devices of the present disclosure allow for the user's respiration to be monitored. For instance, a respiration monitor may detect changes in the circumference of the user's chest that result from the user breathing. The respiration monitor may detect the rate at which the user's chest circumference changes, how much the circumference changes, maximum and/or minimum circumference values, and the like. The data regarding the changes in the user's chest circumference may then be used to determine certain information about the user's respiration. For instance, the user's respiration rate can be determined from the rate at which the user's chest circumference changes. Similarly, how much the user's chest circumference changes and/or the maximum and/or minimum circumference values may be used to approximate the volume of air the user inhales and/or exhales.

The respiration monitor may be secured around at least a portion of the user's chest. As the user breaths, the user's chest circumference increases and decreases. The respiration monitor can detect the changes in the user's chest circumference by detecting changing characteristics or properties of the respiration monitor. For instance, the respiration monitor may have electrical characteristics (e.g., electrical resistance) that change as the respiration monitor expands and contracts. Accordingly, the respiration monitor may expand and contract as the user's chest circumference increases and decreases and in proportion thereto, and the resulting changes in the electrical characteristics of the respiration monitor may be detected. The changes in the electrical characteristics of the respiration monitor may be used to determine the rate at which the user's chest circumference changes, how much the user's chest circumference changes, and/or the maximum and/or minimum circumference values.

The data regarding the one or more physiological conditions, whether detected directly or indirectly, and/or the information derived therefrom (e.g., the volume of air inhaled and/or exhaled) may be used in combination to determine other physiological conditions of the user. For instance, the user's heart rate and the volume of air inhaled and/or exhaled may be used to determine the user's level of oxygen consumption (VO₂). Accordingly, to determine the user's level of oxygen consumption (VO₂), the user simply wears a physiological condition monitor around his chest, which monitors both heart rate and volume of air intake and determines the user's level of oxygen consumption (VO₂) therefrom. No longer is the user required to wear a sensor-equipped mask to detect the volume of air inhaled/exhaled and be connected to a separate heart rate monitor

As noted, the systems and devices of the present disclosure may also include a body motion monitor that monitors certain movements of the user's body. For instance, the body motion monitor may monitor vertical movements of the user's trunk region and/or the user's core body impact. This information may be used to determine an exercise efficiency, such as a running efficiency. The exercise efficiency may indicate, for example, whether the user is running smoothly enough or whether there is too much vertical movement in the user's motion.

Furthermore, the physiological condition monitor may transmit some or all of the collected and/or determined data, via a wireless or wired connection, to a separate electronic device. For instance, the physiological condition monitor may transmit the user's heart rate, respiration rate, and/or oxygen consumption (VO₂) level to a watch worn by the user, a smartphone or other portable electronic device (e.g., PDA, tablet computer) carried by the user, to a local computer (e.g., the user's home computer), or to a remote computer. The separate electronic device may include a display that can present the data to the user.

What is claimed is:

1. A physiological condition monitor, comprising:
a heart rate monitor that monitors a user's heart rate;
a respiration monitor that monitors how much a circumference of the user's chest changes as the user breaths; and
a processor, wherein the processor:
uses the changes in the circumference of the user's chest to approximate the volume of air the user inhales as the user breaths; and
uses the volume approximation and the user's heart rate to determine the user's level of oxygen consumption (VO₂).

2. The physiological condition monitor of claim 1, further comprising one or more straps that selectively secure the physiological condition monitor around the chest of the user.

3. The physiological condition monitor of claim 1, wherein the respiration monitor comprises an elastic material that extends around at least a portion of the circumference of the user's chest.

4. The physiological condition monitor of claim 3, wherein the elastic material comprises rubber.

5. The physiological condition monitor of claim 3, wherein the elastic material is impregnated or doped with at least one of carbon or silicone.

6. The physiological condition monitor of claim 1, wherein the respiration monitor expands and contracts as the user inhales and exhales.

7. The physiological condition monitor of claim 6, wherein an electrical resistance of the respiration monitor is proportional to the length of the respiration monitor.

8. The physiological condition monitor of claim 7, wherein the electrical resistance of the respiration monitor changes as the respiration monitor expands and contracts.

9. The physiological condition monitor of claim 8, wherein the electrical resistance changes in proportion to how much the circumference of the user's chest changes as the user breaths.

10. The physiological condition monitor of claim 1, further comprising a temperature sensor that detects the user's temperature.

11. The physiological condition monitor of claim 10, wherein the processor also uses the user's temperature to determine the user's level of oxygen consumption (VO₂).

12. The physiological condition monitor of claim 1, wherein the heart rate monitor comprises an electrocardiogram (ECG) sensor.

13. The physiological condition monitor of claim 1, wherein the heart rate monitor comprises a light emitting sensor.

14. The physiological condition monitor of claim 1, further comprising a wireless transmitter.

15. The physiological condition monitor of claim 1, further comprising a body motion monitor, the body motion monitor comprising at least one of a pedometer, an accelerometer, and a gyroscope.

16. A physiological condition monitor, comprising:
a heart rate monitor that monitors a user's heart rate;
a respiration monitor that monitors how much a circumference of the user's chest changes as the user breaths, the respiration monitor comprising a stretchable material that has an electrical characteristic that changes as the stretchable material expands and contracts, wherein the changes in the electrical characteristic are proportional to the changes in the circumference of the user's chest; and
a processor, wherein the processor:
uses the changes in the circumference of the user's chest to approximate the volume of air the user inhales as the user breaths; and
uses the volume approximation and the user's heart rate to determine the user's level of oxygen consumption (VO₂).

17. The physiological condition monitor of claim 1, wherein the stretchable material comprises rubber impregnated or doped with at least one of carbon or silicone.

18. A method for determining a person's oxygen consumption (VO₂), the method comprising:
determining a heart rate of the person;
measuring a change in a chest circumference of the person;
approximating a volume of air breathed by the person using the measurement of the change in the chest circumference of the person; and
processing the heart rate and the volume approximation to determine the person's oxygen consumption (VO₂).

19. The method of claim 18, wherein measuring the change in the chest circumference of the person comprises measuring a change in length of a respiration monitor.

20. The method of claim 19, wherein measuring the change in length of the respiration monitor comprises detecting a change in an electrical characteristic of the respiration monitor.
